(19) United States
(12) Patent Application Publication
(10) Pub. No.: US 2005/0002982 A1
(43) Pub. Date: Jan. 6, 2005

(54) Hybrid tissues for tissue engineering

(76) Inventors: David J. Mooney, Ann Arbor, CA (US); Charles A. Vacanti, Uxbridge, MA (US); Craig R. Halberstadt, Charlotte, NC (US); Andrea N. Ferguson, Indianapolis, IN (US); Jennifer J. Marler, Cincinnati, OH (US); Byung-Soo Kim, Seoul (KR)

Correspondence Address: FISH & RICHARDSON PC, 225 FRANKLIN ST, BOSTON, MA, 02110, US

(21) Appl. No.: 10/726,072

(22) Filed: Dec. 1, 2003

Related U.S. Application Data
(63) Continuation of application No. 09/572,786, filed on May 17, 2000, now abandoned.

(30) Foreign Application Priority Data
Nov. 17, 1998 (WO) PCT/US98/24409

(51) Int. Cl. A61K 48/00

(52) U.S. Cl. 424/423; 424/93.21; 424/93.7

(57) ABSTRACT

The present invention comprises an implantable device that provides artificial tissues for repair, augmentation and reconstructive surgery which have mechanical properties comparable to the natural tissues that they supplement or replace. Such devices can be produced by a tissue engineering method comprising seeding a polymer matrix with a first cell type and a second cell type and culturing the seeded matrix under conditions suitable for cell growth or maintenance, whereby a tissue comprising a mixed cell population containing both the first and second cell types is produced. The tissue produced by this method contains a mixed population in which the two cell types are intimately associated without apparent stratification and has mechanical properties which are intermediate between similarly produced tissues containing either one of the two cell types. This invention is particularly useful in forming implantable structural members.

### CROSS-REFERENCE TO RELATED APPLICATIONS

This application is a continuation application of (and claims the benefit of priority under 35 USC 120) U.S. patent application Ser. No. 09/572,786, filed May 17, 2000, which is the national phase of PCT/US 98/24409, filed Nov. 17, 1998, which claims priority to U.S. Provisional Patent Application Ser. No. 60/066,926, filed Nov. 17, 1997.

### FEDERALLY SPONSORED RESEARCH OR DEVELOPMENT

This invention was made with funds pursuant to Grant No. BES-9501376 awarded by National Science Foundation. The federal government has certain rights to this invention.

### BACKGROUND OF THE INVENTION

Contour deformities, whether traumatic, congenital, or aesthetic, generally require invasive surgical techniques for correction. Furthermore, deformities requiring augmentation often necessitate the use of alloplastic prostheses which suffer from problems of infection and extrusion. Engineering new tissues utilizing cell transplantation may provide a valuable tool for reconstructive and plastic surgery applications. Tissue engineering involves the morphogenesis of new tissues from constructs formed of isolated cells and biocompatible polymers. Techniques of tissue engineering employing biocompatible polymer scaffolds have been explored as a means of creating alternatives to prosthetic materials currently used in augmentation and reconstructive surgery.

Chondrocyte transplantation in particular has been successfully used to engineer new tissue masses due to their low metabolic requirements. Cells can be adhered onto a polymeric matrix and implanted to form a cartilaginous structure. This can be accomplished, as described in U.S. Pat. No. 5,041,138 to Vacanti, et al., by shaping of the matrix prior to implantation to form a desired anatomical structure and surgical implantation of the shaped matrix.

Mixtures of dissociated cells and biocompatible polymers in the form of hydrogels have been used to form cellular tissues and cartilaginous structures that include non-cellular material which will degrade and be removed to leave tissue or cartilage that is histologically and chemically the same as naturally produced tissue or cartilage. Slowly polymerizing, biocompatible, biodegradable hydrogels have been demonstrated to be useful as a means of delivering large numbers of isolated cells into a patient to create an organ equivalent or tissue such as cartilage. The gels promote engraftment and provide three dimensional templates for new cell growth.

Unlike the use of solid polymer systems to create a cell-polymer construct, a liquid support matrix that polymerizes to a gel is more easily shaped and molded for custom reconstruction or augmentation. Additionally, a liquid polymer system can potentially be used for injectable delivery, which would be much less invasive than open implantation. Calcium alginate gels have been proposed as a means of delivering large numbers of isolated chondrocytes to promote engraftation and cartilage formation. These initial studies were extended in International Patent Publication No. WO 94/25080 to the formulating of slowly polymerizing calcium alginate gels and to the use of these gels to deliver large numbers of chondrocytes by means of injection, for the purpose of generating new cartilage.

However, whether preformed or injectable implants are used, the engineered cartilaginous tissue can be too rigid for many soft tissue applications. Therefore, there remains a need for new methods of tissue engineering to produce soft tissue analogs.

### SUMMARY OF THE INVENTION

It is an object of this invention to provide artificial tissues for repair, augmentation and reconstructive surgery which have mechanical properties comparable to the natural tissues that they supplement or replace. This and other objects are met by one or more of the following embodiments.

In general, this invention provides a tissue engineering method comprising seeding a polymer matrix with a first cell type and a second cell type; and culturing the seeded matrix under conditions suitable for cell growth or maintenance, whereby a tissue comprising a mixed cell population containing both the first and second cell types is produced. It has been discovered by the present inventors that the tissue produced by this method contains a mixed population in which the two cell types are intimately associated without apparent stratification and has mechanical properties which are intermediate between similarly produced tissues containing either one of the two cell types.

One embodiment of the invention is directed to an implantable structural member for use in treating a patient having an anatomical defect which requires structural support. The defect is treated, at least in part, by providing structural support to adjacent tissue. The structural member is made from a polymeric matrix shaped in the form of the desired support member with a mixture of dissociated cartilage-forming cells and non-cartilage cells deposited on and in the matrix such that when the matrix is implanted, a structural support member is formed. The structural support member has controlled biomechanical properties to provide the required structural support in the area of the defect.

Another embodiment of the invention is directed to a method for treating a patient which has an anatomical defect. The defect is of a type that can be treated, at least in part, by providing structural support to adjacent tissue. The method involves providing a polymeric matrix shaped in the form of a desired support member. A mixture of dissociated cartilage forming cells and non-cartilaginous cells are deposited on and in the matrix to form a matrix/cell construct. The matrix/cell construct is implanted in the patient at a site which needs structural support so that the construct forms a cartilaginous structural member with controlled biomechanical properties to provide the required structural support in the defect area.

The invention is directed to the use of a cartilaginous structural member to provide structural reinforcement to a region of a patient. Surgical procedures and injuries often result in a weakened body structure in a patient. For example, the removal of a diseased or injured organ such as a lung or kidney results in a large cavity in a patient. An implant according to this invention may provide structural support in the cavity left behind after removal of such organs. One advantage of the implant according to this invention it that it is made of a material which is suitably soft to allow a surgeon to rapidly shape and model it during implantation. Further, because of the ability to manufacture implants according to this invention in vitro, a plurality of structures may be prefabricated with a plurality of structural strengths before an operation. The surgeon is thus able to select the most suitable implant in terms of size and structural properties during an operation. Structural properties that may be selected for include structural strength, resistance to bending, twisting and the like.

Another embodiment of the invention is directed to an implant which may be fabricated to allow the mechanical properties of the implant to be specifically tailored for individual applications. The implant according to this invention is capable of providing structural support with mechanical strength depending on the specific need of the location and the patient. For example, an implant according to this invention may be composed of osteoblast and chondrocytes to provide a structural support with a structural strength which exceeds that of natural cartilage. Alternatively, the implant may be composed of chondrocytes and smooth muscle cells to provide a structural strength between than of the two cells types. Implants according to this invention may be manufactured in sheets, columns, fluted columns, polygons, spheres or any complex shape suited to provide structural support in a body cavity. Alternatively, the implant according to this invention may be manufactured in a solid block and shaped before or after seeding by a mixture of chondrocytes and other cells. The shape of the implant may be determined, for example, by CAT scan or MRI imaging of a patient before surgery. Fabrication may be by hand or by computer aided design-computer aided manufacturing (CAD-CAM) systems.

### BRIEF DESCRIPTION OF THE FIGURES

FIG. 1 shows cell density as a function of time in culture for chondrocytes seeded in polyglycolate fibers and for mixed chondrocyte/smooth muscle cells (SMC) on the same substrate.

FIG. 2 shows tissue volume as a function of time in culture for chondrocytes seeded in polyglycolate fibers and for mixed chondrocyte/smooth muscle cells (SMC) on the same substrate.

FIG. 3 shows collagen content of seeded cell mass as a function of time in culture for chondrocytes seeded in polyglycolate fibers and for mixed chondrocyte/smooth muscle cells (SMC) on the same substrate.

FIG. 4 shows elastin content of seeded cell mass as a function of time in culture for chondrocytes seeded in polyglycolate fibers and for mixed chondrocyte/smooth muscle cells (SMC) on the same substrate.

FIG. 5 shows a stress/strain curve for chondrocytes seeded in polyglycolate fibers and for mixed chondrocyte/smooth muscle cells (SMC) on the same substrate.

### DETAILED DESCRIPTION OF THE EMBODIMENTS

This invention has demonstrated that hybrid tissues engineered from chondrocytes and smooth muscle cells reflect mechanical properties intermediate between these two cell types. When a polyglycolate matrix was seeded with equal numbers of chondrocytes and smooth muscle cells, new high density hybrid tissues were formed. The hybrid tissue had an elastic modulus (calculated from mechanical tests) approximately ½ that of chondrocyte alone-derived tissue, indicating that the mechanical properties (and thus feel) of the engineered tissue can be modulated by mixing the two cell types. Importantly, while the collagen content of hybrid tissue was similar to that for tissue containing chondrocytes alone, the tissues containing some smooth muscle contained more elastin at 5 and 8 weeks. While not wishing to be bound thereby, the inventors note that elastin is synthesized in large quantities by smooth muscle cells typically, and the inventors believe that it is likely this alteration of the composition of the engineered tissue that resulted in the control over mechanical properties.

Cell Types

Cell types which may be used for the invention may be selected from any mammalian cell types including epithelial cells, such as adsorptive cells, ciliated cells, and secretory cells; connective tissue cells, such as fibroblast, osteoblast, chondrocytes, and adiopse cells; muscle cells, such as smooth muscle cells, skeletal muscle cells, and cardiac muscle cells; and nerve cells, such as neurons, glial cells, and schwann cells. In essence, a suitable cell support matrix is seeded with predetermined numbers of two or more cell types together, to produce hybrid tissue using normal tissue engineering methods. Typically, the inoculum contains cartilage cells, such as chondrocytes, and smooth muscle cells. One can modulate the properties of chondrocyte-derived tissues by adding a number of other cell types in place of smooth muscle, including adipocytes, skeletal and cardiac muscle, fibroblasts and other soft tissue cells.

The cells in the matrix may be any suitable cell types, but preferably at least a portion of the cells will be derived from the structure to be repaired (i.e., cells of the same cell type will be used). Typically, a portion of the cells are chondrocytes, although osteoblasts or fibroblasts may be used in conjunction with chondrocytes or with other cell types. Chondrocytes may be obtained from any cartilaginous tissue in the patient, or may be allogeneic chondrocytes, so long as care is taken to mitigate any adverse reactions to the allogeneic cells. Additionally, any other cell types known in the field of tissue engineering to proliferate on the matrix of this invention may be used in this method to achieve the desired mechanical properties in the hybrid tissue.

Cells may be isolated from any tissue that comprise chondrocytes. Tissues which may serve as a source for chondrocytes include, for example, cartilage from ribs, nose, ear, joints, unerupted tooth, hyaline cartilage, elastic cartilage and fibrocartilage. Because of the ability to expand an initial chondrocyte population, only a small sample of tissue is required. The tissue may be easily and quickly collected using a biopsy gun with a local anesthetic.

Cartilage forming cells may be isolated according to procedure described in U.S. Pat. No. 5,041,138 which is herein specifically incorporated by reference. Briefly, articulating cartilage was obtained from the shoulders of calves under two weeks of age. The shoulders were washed in Povidone-lodine 10% and the cartilage from the articulating surfaces of the joint were isolated and cut into pieces with dimensions of less than 5 mm per side. Then the cartilage is washed twice in Phosphate Buffered Saline (PBS) with electrolytes and adjusted to neutral pH and incubated at 37° C. in a solution of 0.2% clostridial collagenase (Worthington CLS II, 140 U/mg) and agitated overnight as described by Klagsbrun, (Methods in Enzymology, 58: 560, 1979). This suspension was then filtered using a 153 mg nylon sieve (Tetko, Elmford, N.Y. 10523). The cells were then removed from suspension using centrifugation, washed twice with PBS solution and counted with a hemocytometer. The solution was centrifuged at 1800 rpm and the supernatant above the cell suspension was removed via suction using a micro pipette until the volume of the solution yielded a chondrocyte concentration of 50 million cells per milliliter.

The use of allogeneic cells, and more preferably autologous chondrocytes, is preferred to prevent tissue rejection. However, if an immunological response does occur in the subject after implantation of the implant according to this invention, the subject may be treated with immunosuppressive agents such as, for example, cyclosporin or FKSO6, to reduce the likelihood of rejection of the implant according to this invention. In certain embodiments, chimeric cells, or cells from a transgenic animal, can be seeded onto the polymeric matrix.

Cells obtained for use in the matrix may be used directly or expanded by culture under suitable conditions. Standard cell culture conditions may be used, taking into account that results of this cell expansion process must be suitable for re-introduction into the patient. The cell suspension may contain additives, such as growth factors, colony stimulating factors, cytokines, adhesion peptides, antibiotics, cell nutrients, physiologically compatible buffers and salts, and the like. The components of the cell suspension may be combined using any procedure which preserves viability of a substantial portion of the cells (typically 35% of the cells, preferably at least 50%). Such procedures are known to those skilled in the art of tissue engineering, and suitable procedures are described in the patent publications incorporated herein by reference.

Cells for implantation, including chondrocytes (such as autologous chondrocytes) can be cultured in vitro, if desired, to increase the number of cells available for seeding on the polymeric matrix “scaffold.” Conditions necessary for the successful cultivation of many types of animal cells in vitro are known. Typical culture conditions include the use of buffers and carbon dioxide to buffer media for physiological pH. Nutritional requirements of the cells maybe satisfied by the addition of minerals, amino acids, glucose, and vitamins. Salts, such as KCl and NaCl may be added for physiological osmolality. Serum, such as bovine serum, may be added to provide attachment factors, buffering capacity, essential hormones, lipids, minerals, nutrients, polypeptide growth factors and vitamins. Further, serum may bind and inactivate toxic by-products of cellular metabolism. Supplements such as antibiotics, defined growth factors, and trace elements may be added for the purpose of stimulating growth of cells and inhibiting bacterial contamination.

Many specific media for cell culture have been developed. Examples of media useful for culturing mammalian cells include Eagle's, CMRL, Dulbecco's modified Eagle's, Fischer's, Glasgow, Leibovitz's, McCoy's, F-10, F12, RPMI, Waymouth's, William's, and the like. Formulations of these media are disclosed in numerous publications such as, for example, Gibco BRL Product Catalogue and Reference Guide (Gaithersburg, Md.); incorporated herein by reference. Culture conditions for specific cell types are disclosed, for example, in the mammalian cell catalogue of the American Type Culture Collection (Rockville, Md.).

Cells may be transfected prior to seeding with genetic material. Useful genetic material may be, for example, genetic sequences which are capable of reducing or eliminating an immune response in the host. For example, the expression of cell surface antigens such as class I and class II histocompatibility antigens may be suppressed. This may allow the transplanted cells to have reduced chance of rejection by the host. In addition, transfection could also be used for gene delivery. Chondrocytes could be transfected with specific genes prior to polymer seeding. The cell-polymer construct could carry genetic information required for the long term survival of the host or the tissue engineered neo-organ. For example, cells may be transfected to express insulin for the treatment of diabetes.

Cultures of chondrocytes and other cells may be prepared with or without a cell fractionation step. Cell fractionation may be performed using techniques, such as fluorescent activated cell sorting, which is known to those of skill in the art. Cell fractionation may be performed based on cell size, DNA content, cell surface antigens, and viability. For example, chondrocytes maybe enriched and contaminating cells such as fibroblasts may be reduced. While cell fractionation may be used, it is not necessary for the practice of the invention.

In a preferred embodiment, cells of the same species and preferably immunological profile are obtained by biopsy, either from the patient or a close relative, which are then grown to confluence in culture using standard conditions. If cells that are likely to illicit an immune reaction are used, such as human muscle cells from an immulogically distinct individual, then the recipient can be immunosuppressed as needed, for example, using a schedule of steroids and other immunosuppressant drugs such as cyclosporine. However, in the most preferred embodiment, the cells are autologous. Cells obtained by biopsy are harvested and cultured, and may be passaged as necessary to remove contaminating cells.

Cell support matrices

The matrix material is biocompatible and forms a porous matrix under physiological conditions, typically by cross-linking of biocompatible polymers. The polymers may be natural or synthetic, biodegradable or non-biodegradable, and the polymer(s) may be further modified for enhanced properties. Typical materials for the matrix are described in U.S. Pat. No. 5,041,138, in European Patent No. 0 299 010 or in International Patent Publication No. WO 94/25080, all of which are incorporated herein by reference. The matrix may be a hydrogel, or the matrix may be made up of other materials which form a porous, fibrous network that can contain cells within the contemplation of this invention. Suitable raw materials which may be used to produce a hydrogel in which the cells are suspended include sodium alginate, which has been tested with chondrocytes, as well as PLURONICS™ and TETRONICS™.

The approach provided by this invention will be successful with any number of biomaterials in addition to the polyglycolide used in the Example, including other polyesters, polyanhydrides, and other biocompatible synthetic polymers, as well as naturally-derived materials such as collagen and alginate. One preferred matrix is a polyglycolic acid fiber-based matrix. Methods for the synthesis of the polymers described herein are known to those skilled in the art. See, for example Concise Encyclopedia of Polymer Science and Polymeric Amines and Ammonium Salts, E. Goethals, editor (Pergamen Press, Elmsford, N.Y. 1980). Many polymers, such as poly(acrylic acid), are commercially available.

In a particular embodiment, calcium alginate and/or certain other polymers that can form hydrogels which are malleable are used to encapsulate cells. The hydrogel is produced by cross-linking the polymer, and the polymer solution is mixed with cells to be implanted thereby forming a cell/polymer suspension. The cell/polymer suspension may be injected directly into a patient prior to hardening of the suspension, or the suspension may be hardened in the desired shape prior to implantation into the patient.

The polymeric material used in the implant according to this embodiment is a biocompatible polymer which forms a hydrogel. A hydrogel is defined as a substance formed when an organic polymer (natural or synthetic) is cross-linked via covalent, ionic, or hydrogen bonds to create a three-dimensional open-lattice structure which entraps water molecules to form a gel. Examples of materials which can be used to form a hydrogel include polysaccharides such as alginate, polyphosphazines, and polyacrylates, which are cross-linked ionically, or block copolymers such as PLURONICS™ or TETRONICS™, polyethylene oxide-polypropylene glycol block copolymers which are crosslinked by temperature or pH, respectively. Suitable polymers formulations are described in greater detail in U.S. Pat. No. 5,667,778, incorporated herein by reference.

In general, these polymers are at least partially soluble in aqueous solutions, such as water, buffered salt solutions, or aqueous alcohol solutions, and preferably contain charged side groups, or a monovalent ionic salt thereof. Examples of polymers with acidic side groups that can be reacted with cations are poly(phosphazenes), poly(acrylic acids), poly(methacrylic acids), copolymers of acrylic acid and methacrylic acid, poly (vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups.

Examples of polymers with basic side groups that can be reacted with anions are poly(vinyl amines), poly(vinyl pyridine), poly(vinyl imidazole), and some imino-substituted polyphosphazenes. The ammonium or quaternary salt of the polymers can also be formed from the backbone nitrogens or pendant imino groups. Examples of basic side groups are amino and imino groups.

The water-soluble polymer with charged side groups is crosslinked by reacting the polymer with an aqueous solution containing multivalent ions of the opposite charge, either multivalent cations if the polymer has acidic side groups or multivalent anions if the polymer has basic side groups. Aqueous solutions of the salts of these cations or anions may be added to the polymers to form soft, highly swollen hydrogels and membranes, as described with respect to cations. The biocompatible polymer and the cross-linking agent may be dissolved in any physiologically compatible solvent(s).

For example, alginate can be ionically cross-linked with divalent cations, in water, at room temperature, to form a hydrogel matrix. Due to these mild conditions, alginate has been the most commonly used polymer for hybridoma cell encapsulation, as described, for example, in U.S. Pat. No. 4,352,883 to Lim. In the Lim process, an aqueous solution containing the biological materials to be encapsulated is suspended in a solution of a water soluble polymer, the suspension is formed into droplets which are configured into discrete microcapsules by contact with multivalent cations, then the surface of the microcapsules is crosslinked with polyamino acids to form a semipermeable membrane around the encapsulated materials.

Cell/polymer systems are known which form hydrogel compositions for tissue engineering including suspensions of cells such as chondrocytes in alginate solution to which calcium salts are added to initiate hydrogel formation. Typical systems include chondrocyte/SMC/calcium alginate solutions created by vortexing an isolated cell suspension with sodium alginate solution (e.g., in 0.1M K₂HPO₄, 0.135 M NaCl, pH 7.4) to yield a cellular density of 20×10⁶ cells/ml (a cellular density of approximately 50 percent of that of native articular bovine cartilage) in a 1.0% alginate solution. The chondrocyte/SMC/sodium alginate suspension is stored on ice at 4° C. until use, and prior to injection, 0.2 gm of sterilized CaSO₄ powder is added to each milliliter of the cold chondrocyte/SMC/alginate solution.

Polymers that may be used in implants within the contemplation of this invention also include other natural, synthetic or modified biopolymers which under suitable conditions form hydrogels that are rheologically similar to the hydrogels described herein. Selection of suitable biopolymers is within the skill of the ordinary artisan, in view of the hydrogel characteristics described herein, and suitability may be confirmed based on performance of the polymer in assays and tests described herein. Such polymers must be biocompatible and may be non-biodegradable or biodegradable over a period of days, weeks or even years. Suitable biopolymers include, for example, modified alginates and other modified biopolymers (see, e.g., Putnam A J; Mooney D J (1996), “Tissue engineering using synthetic extracellular matrices,” Nature Medicine, 2(7):824-826, (1996); Mooney, D. J. (1996), “Tissue engineering with biodegradable polymer matrixes,” in Bajpai, Praphulla K (Ed), Proc. South. Biomed. Eng. Conf., 15th, IEEE, NY, 1996; and Wong, W. H., and D. J. Mooney, “Synthesis and properties of biodegradable polymers used as synthetic matrices for tissue engineering, in Atala, et al., eds., Synthetic Biodegradable Polymer Scaffolds (Tissue Engineering), Birkhauser, 1997, ISBN:0817639195).

Levels of individual components in cell polymer compositions described herein can be modified, either singly or in combination, to alter different properties of the formulation both before and after application so as to accommodate particular requirements a) for injection and application, b) for successful engraftment of the implant and creation of required properties and function of the final gel and replacement tissue, or c) for the G12 manufacture, distribution, and application to patients of the formulation. For example, injection of a hydrogel formulation into compact tissues (e.g., muscle, submucosa) requires a high viscosity to prevent extravasation of material. Alterations of viscosity can be achieved by a number of mechanism either singly or in concert, such as (1) selection of the viscosity of the raw material (e.g., low, medium, or high viscosity alginate); (2) concentration of gel (e.g., a range of 0.3% to 3.0% alginate can be used to achieve a broad range of gel viscosities); or (3) amount of highly soluble multivalent cation source to control degree of partial cross-linking.

Producing the hybrid tissue

The cells described above are seeded in a matrix made up of one or more of the polymers described above to produce implants according to this invention. This invention provides artificial tissue compositions comprising one or more biocompatible polymers forming a matrix embedded with a mixed cell population which function as bulk tissue implants having the desired structural and mechanical properties.

Polymeric Matrix Structure

The polymer matrix may be formed before or after the cells are combined with the polymer composition. The polymeric matrix may be fabricated with controlled pore structure as described, for example, in U.S. Pat. No. 5,041,138. The size of the pores maybe used to determine the cell distribution. For example, the pores on the polymeric matrix may be large to enable chondrocytes to migrate to the interior of the structure.

The polymeric matrix may be shaped into any number of desirable configurations to construct an implant to satisfy any number of overall system, geometry or space restrictions. For example, in the use of the polymeric matrix for breast reconstruction, the implant according to this invention may be of a semispherical shape. The polymeric matrix may be made flexible or rigid, depending on the desired final form, structure and function. The implant may be shaped before or after it is seeded with cells. An apparent advantage of using fibrous matrices as described herein is the ease in reshaping and rearranging the structures at the time of implantation.

Seeding

The polymeric matrix may be sterilized using any known method before use. The method used depend on the material used in the polymeric matrix. Examples of sterilization methods include steam, dry heat, radiation, gases such as ethylene oxide, gas and boiling.

Digestive enzymes such as collagenase, trypsin or EDTA can be used to isolate cells that have been cultured to expand the cell population in order to recover the cells for seeding purposes.

The seeding the polymeric matrix with cells may be performed a number of methods which is discussed in U.S. Pat. No. 5,041,138 which is herein specifically incorporated by reference. In one example, braided threads of polyglactin 910, a 90-10 copolymer of glycolide and lactide, coated with polyglactin 370 and calcium stearate (vicryl suture material, Ethicon Co., Somerville, N.J.) were cut into pieces of approximately 17 mm in length. One end was unbraided to expose multiple fibers, 14 microns in diameter. A knot was placed at the other end to aid in locating the polymer during subsequent biopsies. Two polymer fibers were placed into each of 26 Falcon tissue culture dishes, 35 mm in size. Two hundred ml of the above solution was placed on the two fibers in each of 15 wells, thus exposing 30 fibers to the solution containing chondrocytes and keeping 22 polymers free from exposure to chondrocytes to serve as controls. Next, 2 ml of a solution containing Hamm's F-12 culture media and 10% fetal calf serum with L-glutamine (292 mg/cc), penicillin (100 U/cc), streptomycin (100 mg/cc) and ascorbic acid (5 mg/cc) was added to each well. After being incubated at 37° C. for 3, 6, 11, 18, 21 and 28 days, six fibers from each group were examined grossly for the presence and morphologic appearance of chondrocytes using phase contrast microscopy and then evaluated histologically using Hematoxylin and Eosin staining and Aldehyde-Alcian Fuschin stain for chondroitin sulfate, the strongly acidic sulfate of mucopolysaccharides of the cartilage.

The cell-polymer constructs are preferably maintained in vitro to allow for tissue development. Development of the tissue may be measured, at various time points, by monitoring the volume of the tissue that was formed, the density of cells in the tissue, the collagen and elastin content, and the mechanical properties of the developing tissue. The cell density may be higher in the hybrid tissues, for example, due to the presence of smooth muscle cells which will form high density tissues. The volume of the new tissue should be similar, although the tissue volume may vary slightly from the volume for single cell type tissue.

U.S. application Ser. No. 679,177 entitled “Chimeric Neomorphorgenesis of Organs by Controlled Cellular Implantation Using Artificial Matrices” filed Mar. 26, 1991 and U.S. application Ser. No. 933,018 entitled “Chimeric Neomorphorgenesis of Organs Using Artificial Matrices” filed Nov. 20, 1986, and European Patent Publication 0 299 010, all of which are incorporated herein by reference, describe methods and means whereby cells having desired function are grown on polymer scaffold in using cell culture techniques, followed by transfer of the cell polymer scaffold to a patient at a site appropriate for attachment, growth and function after attachment and equal abrasion to produce a functional organ equivalent. Success depends on the ability of the implanted cells to attach to the surrounding environment and to stimulate angiogenesis. Nutrients and growth factors are supplied during cell culture allowing for attachment, survival or growth as needed. U.S. application Ser. No. 933,018 and U.S. application Ser. No. 679,177 disclose several examples of the successful culturing and implantation of hepatocytes, intestine and pancreas cells, with subsequent normal function, including production and secretion of bioactive molecules. Examples of such molecules include growth hormone from pituitary cells, insulin and glucagon from pancreatic cells and clotting factors from liver cells. As described herein, however, there is a need for a different type of function in “organs” which provide primarily a structural function. Examples of types of cells which are useful in these applications include cartilage, bone, and muscle cells.

U.S. application Ser. No. 679,177 describes a technique of placing dispersed cells into synthetic, biodegradable polymer fibers in vitro which have been configured to produce high cell densities by allowing adequate diffusion of nutrients and waste as well as gas exchange. In a preferred method of the present invention, polymer fibers are seeded with mixtures of desired cells, so that the cells attach to the fibers in multiple layers having each subpopulation of cells represented. This technique also allows transplantation of the polymer cell scaffold into animals without disrupting the complex of attached cells. Transplantation of this complex containing a high density of normal-functioning cells with a large surface area into an animal allows the cells to obtain adequate nutrition by diffusion and successful engraftment of functioning tissue, even in the absence of vascularization. It is possible to grow in culture on fibers of biodegradable polymers, mixed populations of cells that individually appear to be morphologically and functionally normal and will proliferate to a cell density sufficient to allow implantation of the cell polymer scaffold in animals and successful engraftment with formation of new tissue equivalent as the polymer resorbs.

Once the cells have begun to grow and cover the matrix, they are implanted in a patient at a site appropriate for the attachment, growth and function. One of the advantages of a biodegradable polymeric matrix is that angiogenic and other bioactive compounds can be incorporated directly into the matrix so that they are slowly released as the matrix degrades in vivo. As the cell polymer structure is vascularized and the structure degrades, the cells will differentiate according to their inherent characteristics.

In the preferred embodiment, the matrix is formed of a bioabsorbable or biodegradable, synthetic polymer, such as a polyanhydride, polyorthoester, or polyglycolic acid. In some embodiments, attachment of the cells to the polymer is enhanced by coating the polymers with compounds, such as basement membrane components, agar, agarose, gelatin, gum arbic, collagens, fibronectin, laminin, gluycosaminoglycans, mixtures thereof, and other materials having properties similar to biological matrix molecules known to those skilled in the art of cell culture. All polymers must meet the mechanical and biochemical parameters necessary to provide adequate support for the cells with subsequent growth and proliferation. Factors, including nutrients, growth factors, inducers of differentiation or the differentiation, products of secretion, immunomodulators, inhibitors of inflammation, regression factors, biologically-active compounds which enhance or allow in growth of the lymphatic network or nerve fibers, and drugs can be incorporated into the matrix or provided in conjunction with the matrix. Similarly, polymers containing peptides such as the attachment peptide RGD can by synthesized for use in forming matrices.

A mesh-like structure formed of fibers which maybe round, scalloped, flattened, star-shaped, solitary or entwined with other fibers is preferred. The polymeric matrix may be made flexible or rigid, depending on the desired final form, structure and function.

Hydrogel matrices

In a preferred embodiment, mixtures of disaggregated chondrocytes, fibroblasts or osteoblasts with other desired cells are suspended in a hydrogel or other liquid/semiliquid carrier and implanted in the patient in need thereof. The cells may be suspended in a liquid/semiliquid carrier which is implanted and then hardens into a cell-containing matrix. The matrix material may be implanted in the fluid state to conform to the desired shape and then cure, crosslink or harden to form the matrix, or the matrix may be formed first and then implanted.

Procedures for preparing the matrices and seeding them with cells are described in the publications incorporated herein by reference, and the skilled worker will readily adapt those procedures to this invention in view of the guidance provided herein. The hydrogel-cell suspension may be prepared as described for products used in treatment for vesicoureteral reflux using autologous auricular chondrocytes in sodium alginate. Alternatively, the hydrogel-cell suspension may be prepared as described in International Patent Publication No. WO 97/17038, by Vacanti, et al., entitled “Hydrogel-cell Composition—for Generating New Tissue on Surface of Structure or Organ,” incorporated herein by reference.

As described herein, an injectable biodegradable polymer as a delivery vehicle for mixed cells to produce a hybrid tissue is useful in the treatment of reflux and incontinence. The cell-containing suspension can be injected through a cystoscopic needle, having direct visual access with a cystoscope to the area of interest, such as for the treatment of vesicoureteral reflux or urinary incontinence. The suspension can also be applied to reconstructive surgery, or applied anywhere in the human body where a biocompatible permanent injectable material is necessary. The suspension can be injected indiscoptically, for example through a laryngoscope for injection into the vocal cords for treatment of dysphonial or through a hysteroscope for injection into the fallopian tubes as a method of rendering the patient infertile, or through a proctoscope, for injection of the substance in the perirectal sphyncter area, thereby increasing the resistance in the sphyncter area and rendering the patient continent of stool. The suspension can be injected via a syringe and needle directly into a specific area wherever a bulking agent is desired, i.e., a soft tissue deformity such as that seen with areas of muscle atrophy due to congenital or acquired diseases or secondary to trauma, bums, and the like. An example of this would be an injection of the suspension in the upper torso of a patient with muscular atrophy secondary to nerve damage. The suspension can also be injected as a bulking agent for hard tissue defects such as bone or cartilage defects, either congenital or acquired disease states, or secondary to trauma, bums or the like. An example of this would be injection into the area surrounding the skull where a bony deformity exists secondary to trauma. The injection in these instances can be made directly into the needed area with the use of a needle and syringe under local or general anesthesia. This suspension could be also be injected percutaneously by direct palpation, such as by placing a needle inside the vas deferens and occluding the same with the injected bulking substance, thus rendering the patient infertile. The suspension could also be injected through a catheter or needle with fluoroscopic, sonographic, computed tomographic, magnetic resonance, imaging or other type of radiologic guidance. This would allow for placement or injection of this substance either vascular access or percutaneous access to specific organs or other tissue regions in the body, wherever a bulking agent would be required. Further, the substance could be injected through a laparoscope or thoracoscope to any intraperitoneal or extraperitoneal or thoracic organ. For example, the suspension could be injected in the region of the gastro-esophageal junction for correction of gastro-esophageal reflux. This could be performed with either a thoracoscope injecting the substance in the esophageal portion of the gastro-esophageal, or via laparoscope by injecting in the gastric portion of the gastro-esophageal region or by a combined approach.

### EXAMPLES

In order to facilitate a more complete understanding of the invention, an Example is provided below. However, the scope of the invention is not limited to specific embodiments disclosed in the Example, which is for purposes of illustration only.

### Example 1

Tissue Produced by Co-culture of Two Cell Types

In this study, rat aortic smooth muscle cells (SMCs) and pig articular chondrocytes were co-cultured on fiber-based polyglycolic acid (PGA) matrices (5×5 mm, 2-mm thick) to address this hypothesis. In essence, equal numbers of chondrocytes and smooth muscle cells were seeded together onto a polyglycolic acid fiber-based matrix or chondrocytes alone, as per normal methods. Cells were seeded by agitating the polymer matrices and a cell suspension in 50 ml centrifuge tubes with an orbital shaker. After seeding, cell-polymer constructs were cultured in stirred bioreactors for 8 weeks. The cell-polymer constructs were then maintained in vitro for eight weeks to allow for tissue development.

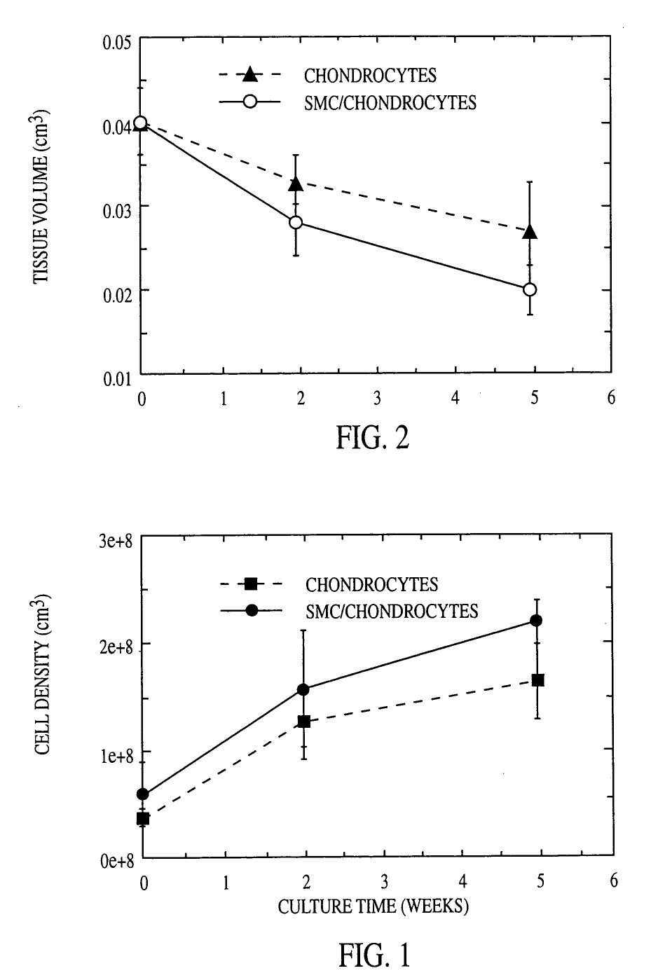

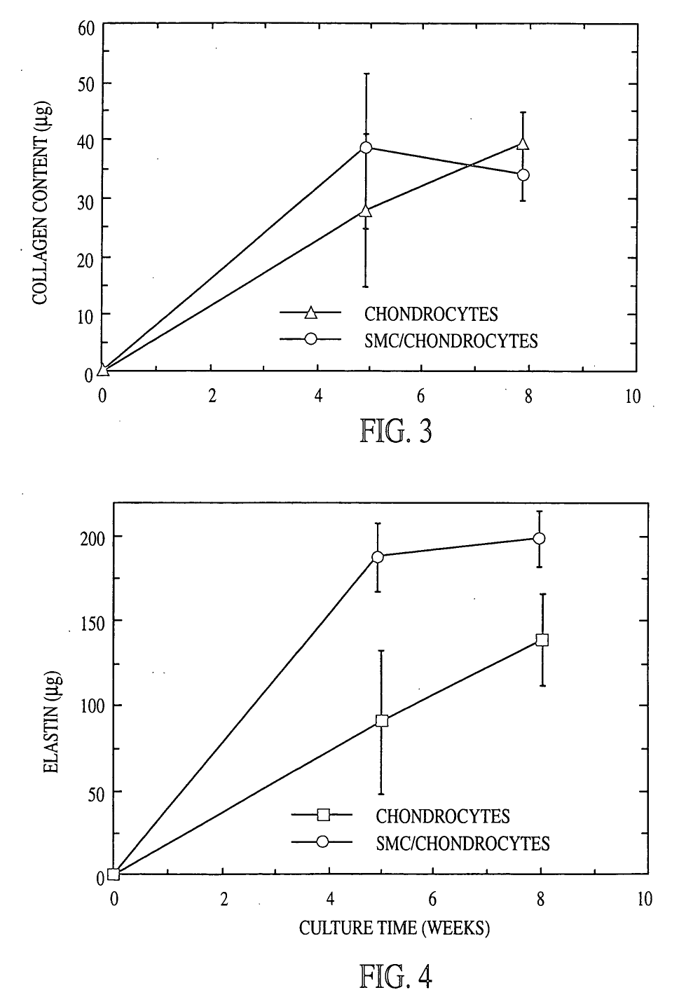

At various time points, measurements were made of the volume of the tissue that was formed, the density of cells in the tissue, the collagen and elastin content, and the mechanical properties. The data is shown in FIGS. 1-5. In essence, in both cases new high density tissues formed. The engineered tissue approximately maintained the original size and shape of the polymer matrices. As shown in FIG. 1, the cell density was higher in the hybrid tissues, likely due to the presence of smooth muscle cells which will form high density tissues. As shown in FIG. 2, the volume of the new tissue was similar, although slightly greater in the chondrocyte alone tissue. Importantly, while the collagen content of the new tissue was similar (FIG. 3), the tissues containing smooth muscle contained more elastin at 5 and 8 weeks (FIG. 4).

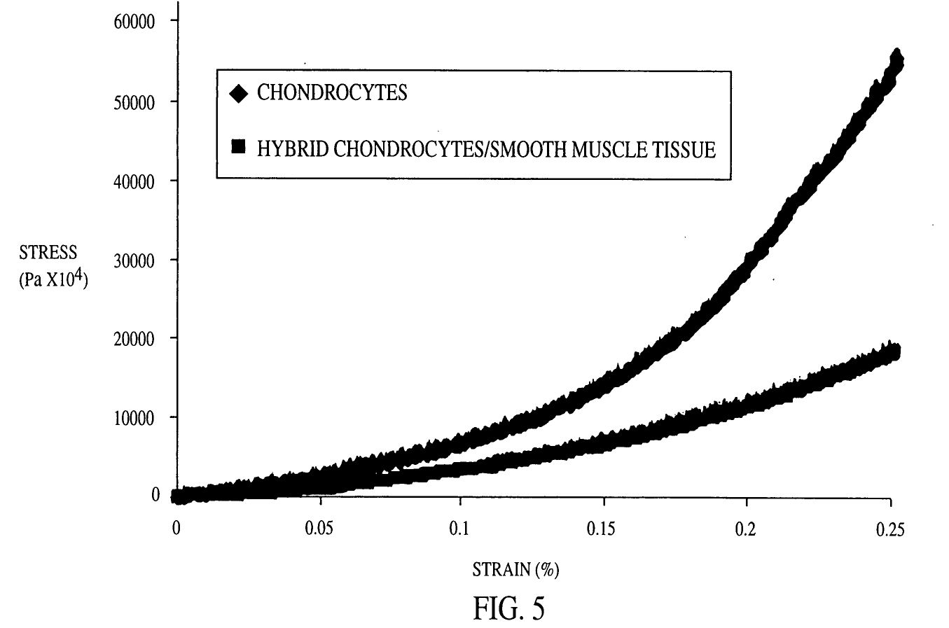

FIG. 5 shows typical results for mechanical testing of the two types of tissues. The hybrid tissue had an elastic modulus (calculated from these mechanical tests) approximately ½ that of the chondrocyte alone-derived tissue, in support of our initial hypothesis that we could modulate the mechanical properties (and thus feel) of the engineered tissue by mixing the two cell types. Mechanical testing with a mechanical testing system showed the compressive modulus of the engineered cartilage to be 71±10 kPa and hybrid tissues 36±5 kPa. This difference in modulus indicated that the cartilaginous construct had become softer by addition of the smooth muscle element. Histology of the new tissues showed elastin deposition and general cellularity which was consistent with the quantitation in these graphs. This approach may be useful to engineer tissues for a variety of reconstructive surgery applications.

For purposes of clarity of understanding, the foregoing invention has been described in some detail by way of illustration and example in conjunction with specific embodiments, although other aspects, advantages and modifications will be apparent to those skilled in the art to which the invention pertains. The foregoing description and examples are intended to illustrate, but not limit the scope of the invention. Modifications of the above-described modes for carrying out the invention that are apparent to persons of skill in medicine, immunology, hybridoma technology, pharmacology, and/or related fields are intended to be within the scope of the invention, which is limited only by the appended claims.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

1. A cell-containing implant for implantation into a patient comprising:
a polymeric matrix shaped in the form of the desired implant; and
a mixture of dissociated cells comprising smooth muscle cells and cells of at least one other cell type deposited on and in the matrix.

2. The cell-containing implant of claim 1, wherein the cells of at least one other cell type are chondrocytes or osteoblasts, and further wherein the implant provides structural support with a structural strength that exceeds that of natural cartilage.

3. The cell-containing implant of claim 1, further comprising an angiogenic compound.

4. The cell-containing implant of claim 1, wherein the polymeric matrix comprises polymers coated with agar, agarose, gelatin, gum arabic, or a mixture of two or more of these substances.

5. An implantable structural member for use in treating a patient having an anatomical defect that requires structural support, the structural member comprising
(a) a polymeric matrix shaped in the form of the desired support member; and
(b) a mixture of cells containing at least dissociated cartilage-forming cells and non-cartilage cells, wherein the non-cartilage cells comprise smooth muscle cells deposited on and in the matrix,
such that when the matrix is implanted, a structural support member is formed.

6. The implantable structural member of claim 5, wherein the matrix is in the form of a sheet, a column, a fluted column, a polygon, or a sphere.

7. The implantable structural member of claim 5, wherein the matrix comprises polyglycolide, polylactide, collagen, alginate, or a mixture thereof.

8. The implantable structural member of claim 5, further comprising an angiogenic compound.

9. The implantable structural member of claim 5, wherein the polymeric matrix comprises polymers coated with agar, agarose, gelatin, gum arabic, or a mixture of two or more of these substances.

10. A tissue engineering method comprising
seeding a polymer matrix with a mixture containing smooth muscle cells and cells of at least one other type; and
culturing the mixture of cell types under conditions suitable for cell growth or maintenance,
whereby a hybrid tissue is formed comprising a mixed cell population containing at least the smooth muscle cells and the cells of at least one other type.

11. The tissue engineering method of claim 10, wherein expanded populations of the smooth muscle cells and the cells of at least one other type are produced by culturing after both types of cells are seeded onto the matrix.

12. The tissue engineering method of claim 10, wherein expanded populations of the smooth muscle cells and the cells of at least one other type are produced before seeding unto the matrix.

13. The tissue engineering method of claim 10, wherein the cells of the at least one different cell type are chondrocytes or osteoblasts, and further wherein the hybrid tissue provides structural support with a structural strength that exceeds that of natural cartilage.

14. The tissue engineering method of claim 10, wherein the matrix is in the form of a sheet, a column, a fluted column, a polygon, or a sphere.

15. The tissue engineering method of claim 10, wherein the matrix comprises polyglycolide, polylactide, collagen, alginate, or a mixture thereof.

16. A method for treating a patient having an anatomical defect of a type that can be treated, at least in part, by providing structural support to adjacent tissue, said method comprising the steps of:
providing a polymeric matrix shaped in the form of a desired support member, the polymer matrix having deposited thereon a mixture of dissociated cells comprising smooth muscle cells and cells of at least one other cell type to produce a matrix/cell construct; and
implanting the matrix/cell construct in the patient at a site that needs structural support so that the construct forms a structural member with controlled biomechanical properties to provide the required structural support in the defect area.

17. The method of claim 16, wherein the cells of the at least one other cell type include cartilage-forming cells and the construct forms a cartilaginous structure member.

18. The method of claim 16, wherein the shape of the polymer matrix is determined by CAT scan or MRI imaging of a patient before surgery.

19. The method of claim 16, wherein the polymer matrix is manufactured in a solid block and altered in shape before or after seeding with a mixture of cells.

20. The method of claim 19, wherein the shape of the polymer matrix is fabricated by hand or by computer aided design-computer aided manufacturing (CAD-CAM) systems.
